(19) United States
(12) Patent Application Publication
(10) Pub. No.: US 2007/0207066 A1
(43) Pub. Date: Sep. 6, 2007

(54) AIR STERILIZATION DEVICE AND USES THEREOF

(75) Inventors: Charles Thur, Broadview Heights, OH (US); David Brickner, Concord, OH (US); Richard C. Farone, Willoughby, OH (US)

Correspondence Address: FAY SHARPE LLP, 1100 SUPERIOR AVENUE, SEVENTH FLOOR, CLEVELAND, OH, 44114, US

(73) Assignee: GUARDIAN TECHNOLOGIES LLC, Bedford Hts., OH (US)

(21) Appl. No.: 11/680,958

(22) Filed: Mar. 1, 2007

Related U.S. Application Data
(60) Provisional application No. 60/778,180, filed on Mar. 2, 2006.

(51) Int. Cl. A61L 9/20

(52) U.S. Cl. 422/121; 96/224; 96/222

(57) ABSTRACT

An air sterilizer for eliminating an associated airborne contaminant from an associated volume of air is provided. The air sterilizer includes a housing including an air inlet and an air outlet, an airflow pathway initiating approximately at the air inlet of the housing and terminating approximately at the air outlet of the housing, and an ultra-violet (UV) radiation source for producing a germicidal UV light. The UV source is disposed in the airflow pathway of the housing for exposing the associated airborne contaminant to the germicidal UV light. The air sterilizer further includes a reflecting member for increasing the exposure of the germicidal UV light to the associated airborne contaminant in the airflow pathway. The reflecting member being disposed in the housing proximal to the UV source. A power supply is provided for powering the UV source. The power supply is disposed within the housing and an electrical power plug assembly is secured to the housing and is in electrical communication with the power supply. The power plug assembly is selectively engageable with an associated electrical power receptacle and capable of supporting the air sterilizer when the power plug is selectively engaged with the associated electrical power receptacle.

A claim for domestic priority is made herein under 35 U.S.C. §119(e) to U.S. Provisional App. Ser. No. 60/778,180 filed on Mar. 2, 2006, the entire disclosure of which is incorporated herein by reference.

### BACKGROUND

The present invention relates to an air sterilization device and uses thereof. More specifically, the present invention relates to a wall mountable air sterilization device that employs ultraviolet radiation to remove airborne contaminants (e.g., microorganisms) from an environment in which the device is positioned.

Air contamination, particularly indoor air contamination, contributes to human health complications. Specifically, airborne chemical and/or biological contaminants, particularly when present in poorly ventilated areas, cause a wide variety of human illnesses. Example chemical contaminants include, by way of non-limiting example, formaldehyde, aerosols, toluene, hydrocarbons, carbon monoxide, and the like, and are known to cause such health complications as eye irritation, headaches, nose and/or mucosal irritation, fatigue and the like. Example biological contaminants include, by way of non-limiting example, bacteria, fungi, fungi spores, protozoa, viruses, algae, pollen, various antigenic agents, and the like, and are known to cause such health complications as pneumonia, fever, mycotoxicosis, various infections, asthma and the like.

Prior art air sterilization devices suffer from a number of problems. First, the devices are large and consume significant amounts of useable space in an office or home environment. Second, though they may provide a means for reducing airborne contaminants, they do not address the odors that may be present in the areas of intended use. In addition, the prior art devices are very inefficient. More often than not, they require an associated volume of air in the area of intended use to be recirculated multiple times through the device in order for the contaminants to be effectively removed.

The air sterilization device of the present invention is particularly configured to overcome one or more of the aforementioned problems in removing and/or generally reducing the presence of air borne contaminants to thereby provide a more healthy and less infection-prone environment.

### SUMMARY OF THE INVENTION

According to one aspect of the present invention, an air sterilizer for eliminating an associated airborne contaminant from an associated volume of air is provided. The air sterilizer includes a housing including an air inlet and an air outlet, an airflow pathway initiating approximately at the air inlet of the housing and terminating approximately at the air outlet of the housing, and an ultra-violet (UV) radiation source for producing a germicidal UV light. The UV source is disposed in the airflow pathway of the housing for exposing the associated airborne contaminant to the germicidal UV light. The air sterilizer further includes a reflecting member for increasing the exposure of the germicidal UV light to the associated airborne contaminant in the airflow pathway. The reflecting member being disposed in the housing proximal to the UV source. A power supply is provided for powering the UV source. The power supply is disposed within the housing and an electrical power plug assembly is secured to the housing and is in electrical communication with the power supply. The power plug assembly is selectively engageable with an associated electrical power receptacle and capable of supporting the air sterilizer when the power plug is selectively engaged with the associated electrical power receptacle.

According to another aspect of the present invention, a compact wall mountable air sterilization device for eliminating an associated airborne contaminant from an associated volume of air is provided. The air sterilization device is capable of being supported by an associated household electrical outlet. The air sterilization device includes a body including a front, a rear, a top, an air inlet and an air outlet. An airflow pathway initiates approximately at the air inlet of the body and terminates approximately at the air outlet of the body. An ultra-violet (UV) radiation source is provided for producing a germicidal UV light. The UV source is disposed in the airflow pathway of the body for exposing the associated airborne contaminant to the germicidal UV light. A tube reflector is disposed about the UV source for increasing the exposure of the germicidal UV light to the associated airborne contaminant. The tube reflector forms at least a portion of the airflow pathway. A fan is disposed within the airflow pathway for accelerating the flow of the associated volume of air through the body. A power supply is provided for powering the UV source and the fan. The power supply is disposed at the bottom of the housing. A rotatable electrical power plug is secured to the body and is in electrical communication with the power supply. The power plug is selectively engageable with the associated household electrical outlet and is capable of supporting the body when the power plug is selectively engaged with the associated household electrical outlet.

According to yet another aspect of the present invention, a compact wall mountable ultra-violet (UV) sterilizer for eliminating an associated airborne microorganism from an associated volume of air is provided. The UV sterilizer is capable of being supported by an associated household electrical outlet. The UV sterilizer includes a housing including an air inlet and an air outlet. An airflow pathway initiates approximately at the air inlet of the housing and terminates approximately at the air outlet of the housing. An ultra-violet (UV) radiation source is provided for producing a germicidal UV light. The UV source is disposed in the airflow pathway of the housing for exposing the associated airborne microorganism to the germicidal UV light. A tube reflector is provided for increasing the exposure of the germicidal UV light to the associated airborne microorganism in the airflow pathway. The tube reflector is disposed in the housing proximal to the UV source and includes a surface for receiving a UV activated layer of titanium dioxide (TiO₂). A fan is disposed within the airflow pathway for accelerating the flow of the associated volume of air through the housing. A power supply for powering the UV source and the fan is provided and disposed within the housing. A rotatable electrical power plug is secured to the housing and is in electrical communication with the power supply. The power plug is selectively engageable with an associated household electrical outlet and capable of supporting the air sterilizer when the power plug is selectively engaged with the associated household electrical outlet. And, a scent receptacle is defined in the housing adjacent the air outlet for receiving a scented member for dispersing a fragrance into the associated volume of air when the air sterilizer is in an on state.

### BRIEF DESCRIPTION OF THE DRAWINGS

These and other features and a more thorough understanding of the present invention may be achieved by referring to the following description, taken in conjunction with the accompanying drawings, wherein:

FIG. 1 is a perspective view of a first embodiment of an air sterilization device, according to the present invention.

FIG. 2 is a front elevation view of the air sterilization device of FIG. 1.

FIG. 3 is a side elevation view of the air sterilization device of FIG. 1.

FIG. 4 is cross sectional view of the air sterilization device along the a plane 4-4 shown in FIG. 2, illustrating an airflow pathway by which air is drawn into the device, treated and subsequently expelled.

FIG. 5 is an exploded view of the air sterilization device of FIG. 1, illustrating, among other items, a UV radiation source, a tube reflector, a power plug assembly and a scent dispersing member.

FIG. 5A is a detailed exploded view of the power plug assembly of FIG. 5.

### DETAILED DISCUSSION OF EXAMPLE EMBODIMENTS

Disclosed according to the present invention is an air sterilization device and uses thereof. As will be more fully explained hereinafter, the air sterilization device of the present invention includes a source of ultraviolet (“UV”) radiation that, when exposed to a stream of air, exhibits a germicidal effect for removing airborne contaminants therefrom. As will also be more fully explained, the sterilization device is adapted to be mounted directly to a wall outlet, thereby reducing the device's footprint and eliminating the need for placing the device on a surface, such as a countertop, a floor and the like.

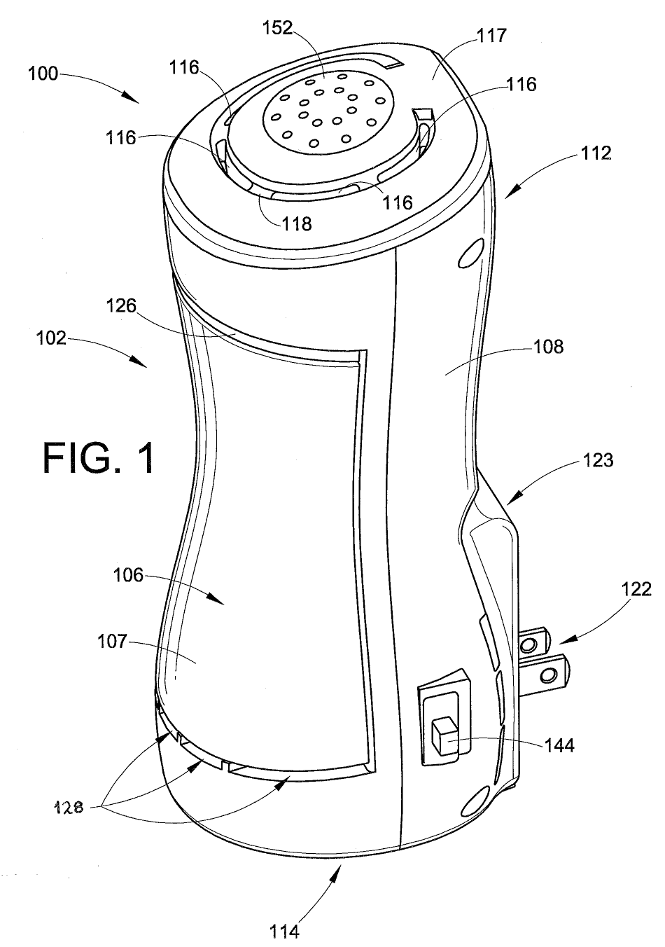

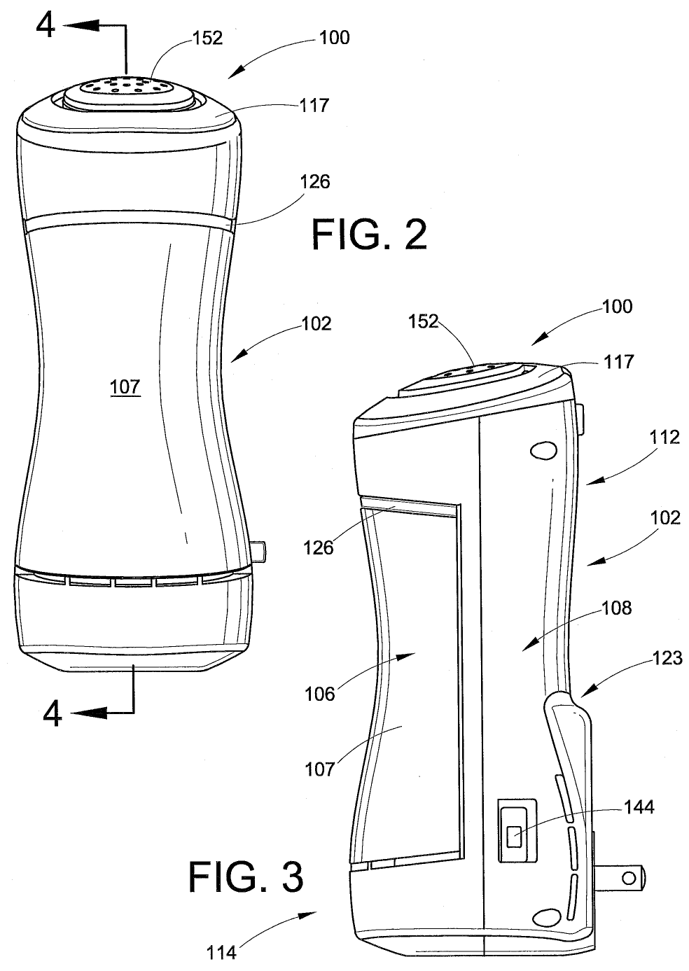

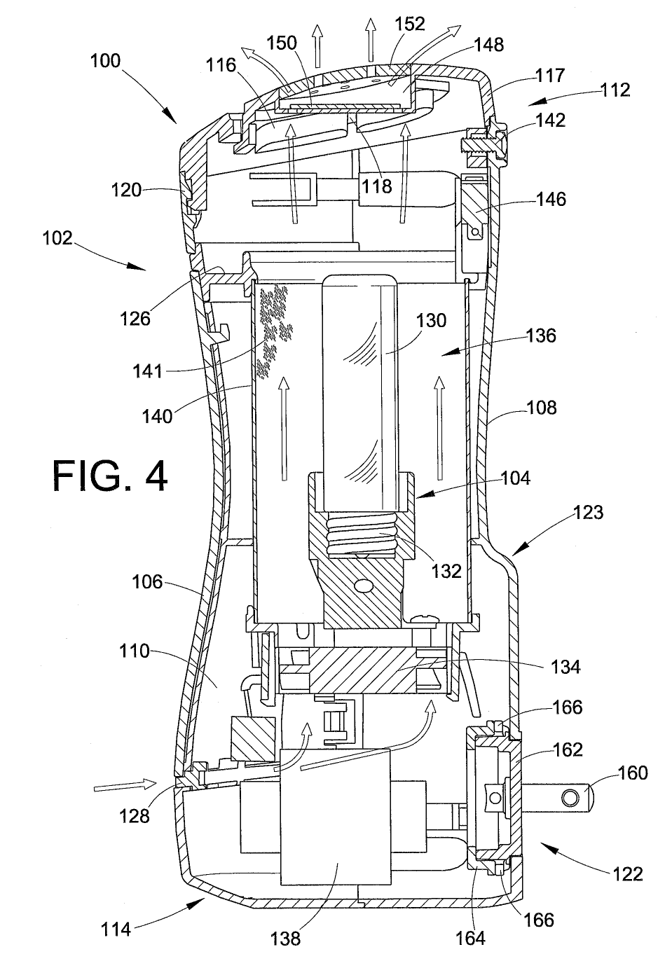

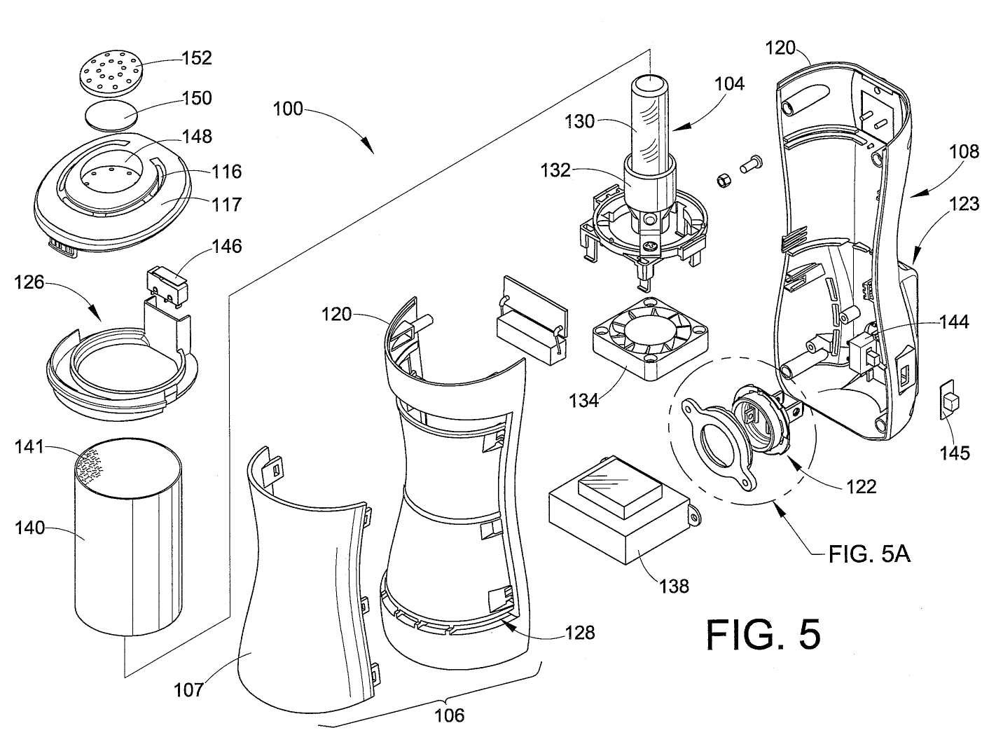

Turning now to FIGS. 1-5, a first embodiment of an air sterilization device 100 is shown according to the present invention. The device 100 generally includes a housing 102, a UV radiation source 104 (FIG. 4) maintained generally within the housing 102, and various control elements that direct and/or enable operation of the device 100.

Turning now to a discussion of the housing 102, the housing 102 is provided as a generally cylindrically elongate body adapted to be vertically or horizontally disposed, thereby orienting its longitudinal axis along a vertical plane or horizontal plane, respectively. The housing 102 houses the UV radiation source 104 and operates as a conduit through which a stream of air can be fluidicly passed for treatment by the UV radiation source 104. More specifically, and as will be more fully explained, the housing 102 generally includes an air inlet or vent through which a contaminated stream of air is drawn into an inner cavity of the housing 102, and wherein the contaminated stream or airflow is exposed to the UV radiation. The housing 102 generally also includes an air outlet or exhaust vent through which the UV-exposed or otherwise decontaminated airflow returns to the external environment subsequent to such UV exposure. The flow path between the air inlet and air outlet generally defines an air flow pathway, as indicated by the flow arrows in FIG. 4.

With continued reference to FIG. 1, the housing 102 can be generally defined by a front portion 106, a rear portion 108 and an inner cavity 110. The front portion 106 generally defines a front or forward half of the device 100 and serves as an interface of the device 100 with its external environment. The front portion 106 includes a front panel 107 that can serve as a substrate for the addition of various aesthetic features or graphic indicia such as a brand, a logo and the like. The rear portion 108 is generally disposed at the rear or wall facing portion of the device 100 and is spaced apart from the front portion 106 except where the rear portion 108 is joined to the front portion 106. As previously mentioned, the device 100 can be configured in a generally cylindrical manner, so the front and rear portions 106, 108 are, by extension, generally also cylindrical and generally continuous surfaces.

With reference to FIG. 4, the housing 102 inner cavity 110 can be generally defined as a space disposed within the housing 102 between the front and rear portions 106 and 108. As will be more fully explained hereinafter, the inner cavity 110 provides the conduit through which the airflow passes, and is accordingly communicatively associated with the device 100 air inlet and outlets. As will also be more fully explained, the UV radiation source 104 can be also disposed at least partially within the inner cavity 110 for posting the UV radiation source 104 in proximity to the airflow pathway.

The housing 102 is also characterized by an upper (per the orientation depicted in the figures) portion 112 and a lower (per the orientation depicted in the figures) portion 114. The upper portion 112 is generally disposed supra relative to the lower portion 114 when the device 100 is positioned along a wall. The upper portion 112 generally includes the previously mentioned air outlet 116 which operates to permit a UV-exposed airflow to escape from the device 100 inner cavity 110 and thereby return decontaminated and/or sterilized air to the environment of the device 100.

The air outlet 116 may be provided in any suitable configuration, with regard given to the aims of the present invention. FIGS. 1-5 illustrate the air outlet 116 configuration in which the air outlet 116 is provided as a generally cylindrical member adapted to be disposed at least partially within the housing 102 upper portion 112. Furthermore, the housing 102 upper portion 112 includes a shoulder 120 upon which the air outlet 116 is disposed. More specifically, the shoulder 120 can be defined as, relative to an inner wall surface of the housing 102, as a circumferentially and/or diametrically reduced portion, with the remainder of the inner wall surface extending below the shoulder 120. This configuration creates a slot-like feature that permits the air outlet 116 to be slid in the housing 102 and maintained at a terminal position.

With further reference to the air outlet 116, the air outlet 116 can be provided as a unitary structure associable with the housing 102. The air outlet 116 can be defined by a circumferential top body portion 117, which extends generally parallel to the longitudinal axis of the housing 102, and a plurality of ribs 118 extending therebetween. The ribs 118 are each generally prismatically rectangular structures extending across the gap or opening provided by the air outlet 116 along the top 117. Additionally, the ribs 118 can be each outwardly angularly offset relative to the air outlet 116 of the top 117. An outward angular offset configuration of the ribs 118 can suitably direct the outgoing airflow away from the device 100 and away from the rear portion 108 of the device 100. The outward angular offset configuration, depending on the particular angle of offset, may suitably also provide a safety feature that blocks and/or prevents a direct line of sight to the UV radiation source 104, as will be more fully explained hereinafter.

The air outlet 116 is associated with the device 100 housing 102 in any suitable manner. For example, a suitable adhesive, particularly one suitable for exposure to long durations of elevated temperatures, may be disposed at the upper portion 112 between the front and rear portions 106, 108 of the housing 102 and the top 117, or in any other suitable arrangement. By way of additional example, a mechanical fastener, such as a rivet, a bolt, a staple, or the like, may be employed to retain the air outlet 116 relative to the housing 102. In another aspect, various means may be employed to permit the air outlet 116 to be removably associable with the housing 102, such as a snap lock features or the like. Such removability may suitably provide advantageous in connection with replacing a bulb associated with the UV radiation source 104, as will be more fully explained hereinafter. Despite the foregoing, it is to be appreciated that the air outlet 116 may suitably be a structure integral with and/or unitary to the housing 102.

The housing 102 upper portion 116 may suitably also include one or more baffles disposed within the housing 102 inner cavity 110. The baffles may provide a number of benefits relative to the device 100. In one regard, the baffles may each extend from opposite portions of the inner wall surface of the housing 102 and can be of a dimension that causes them to at least slightly overlap along a lateral midpoint of the inner cavity 110. In such a configuration, the baffles suitably provide a retardant to airflow at, above, or below the UV radiation source 104. This retardant suitably forces the airflow in the inner cavity 110 to increase its dwell time or duration of exposure to the UV radiation source 104 and effectively increase the extent of germicidal activity. It is to be appreciated that the housing 102 may include additional baffles or other reversing structures for increasing the dwell time that the contaminated airflow remains in the housing 102. In another regard, the baffles suitably also provide a barrier to visual contact with the UV radiation source 104. Similar to the ribs 118 in the air outlet 116, the baffles can be arranged in an overlapping configuration to provide a physical impediment that prevents accidental or other visual contact with the UV radiation source.

The device 100 housing 102 can be also defined by the lower portion 114. In one aspect, the lower portion 114 provides a site from which the powerplug assembly 122 extends. Moreover, a portion of the powerplug assembly 122 extends through or from a rear surface of the rear portion 108 of the device 100. The rear surface is defined as such because it remains in proximity to a wall when the device 100 is associated with a wall outlet. In another embodiment, the lower portion 114 includes a projection 123 that extends from the housing 102 generally transversely relative to its longitudinal axis, and which projection 123 includes a portion of the powerplug assembly 122 thereby disposing the powerplug assembly 122 away from the rear portion 108 of the housing 102. In this manner, the projection 123 distances the housing 102 when the device 100 is associated with a wall outlet.

The housing 102 lower portion 114 suitably also includes the air inlet 128. The air inlet 128 can be oriented in general opposition to the powerplug assembly 122 and provides an orifice through which air can be drawn from the external environment into the device 100 inner cavity 110 for exposure to the UV radiation source 104 and subsequent return to the external environment.

The device 100 also includes a fan 134 disposed at least partially within the housing 102 inner cavity 110, and relatively above the air inlet 128. The fan 134, when active, operates to forcefully draw air into the air inlet 128 from the external environment. As air is drawn into the inner cavity 110, the fan 134 then drives the air along the UV radiation source 104, and subsequently through the air outlet 116, as generally depicted in FIG. 4.

Turning now to a discussion of the UV radiation source 104, the sterilization device 100 of the present invention includes the UV radiation source 104 for irradiating an airstream and removing contaminants (e.g., microorganisms, etc.) therefrom. In general, the UV radiation source 104 is provided as a source capable of producing germicidal ultraviolet radiation; however, it is to be appreciated that any device, either alone or in combination with a UV radiation source, may be employed having germicidal capabilities (e.g., an ozone generator and the like).

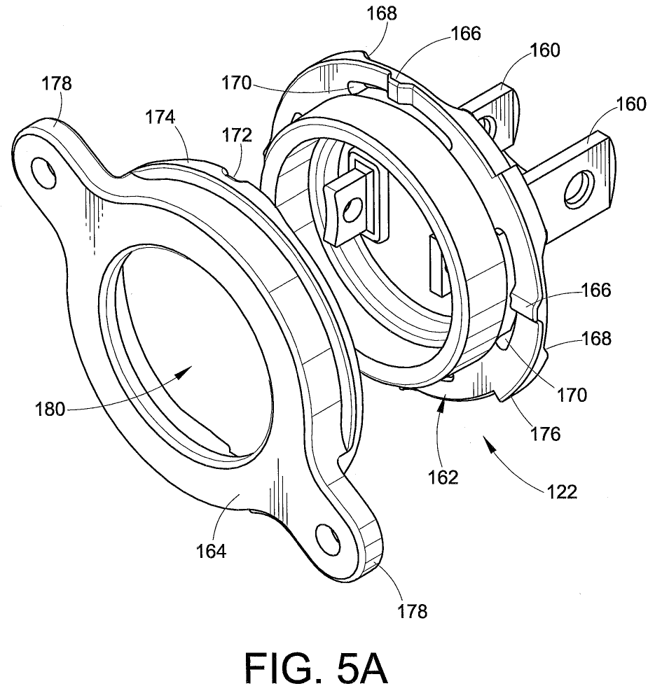

With reference to FIGS. 4 and 5, the UV radiation source 104 includes a bulb 130 and a socket 132, both generally maintained and/or disposed in a germicidal chamber 136. The bulb 130 is a generally conventional bulb capable of emitting ultraviolet radiation, although any suitable bulb may be employed, such as a cold cathode fluorescent bulb or a light emitting diode (LED). The bulb 130 is configured to emit any suitable type of UV radiation, such as UV-A, UV-B, UV-C, or may suitably be configured to simultaneously emit two or more of the foregoing. In one embodiment, the bulb 130 can be configured to emit UV-C radiation, radiation having a wavelength of approximately 200-280 nanometers (nm), even more radiation having a wavelength of approximately 254 and/or 253.7 nm. However, it is to be appreciated that any suitable wavelength may be employed, and the present invention is not to be construed as limited to the presently disclosed exemplary wavelengths. The capabilities of the bulb are selected on its ability to penetrate microorganism outer structures (e.g., cell wall, plasma membrane, etc.), and cause DNA mutations and/or modifications that interfere with cellular replication, thereby leading to cellular death and air sterilization.

The bulb 130 may include other aspects that improve its performance characteristics. For example, the bulb 130 may include a high purity synthetic quartz glass envelope. It may suitably also include a clear coating on the inside for decreasing depreciation of UV-C radiation output (so called “solarization”). By way of additional example, the bulb 130 may be configured to produce Ozone (O₃).

The UV radiation source 104 generally also includes the socket 132 for maintaining the bulb 130 in a desired orientation and for communicating current to the bulb 30. The socket 132 is a generally conventional socket providing these capabilities, and is further coupled to a power supply, transformer, or a ballast 138 or similar feature capable of generating, transferring and/or converting current for use by the bulb 130.

As previously mentioned, the UV radiation source 104, defined generally as the bulb 130 and socket 132, can be disposed within the germicidal chamber 136. In general, the germicidal chamber 136 can be disposed within the housing 102, and generally along a longitudinal midportion thereof, between the fan 134 and the baffles. Further, the socket 132 can be maintained within the germicidal chamber 136 spaced apart from the housing 102 inner wall surface, disposing the bulb 130 upright and generally parallel to the longitudinal axis of the housing 102. Additionally, the spaced apart disposition of the socket 132 generally positions the socket 132 along a midpoint of the inner cavity 110 (relative to the inner wall surface), thereby enabling the airstream to generally envelope the bulb 130 as it flows therepast.

The germicidal chamber 136 may suitably include various materials disposed along the housing 102 inner wall surface for increasing the effectiveness of the UV radiation source 104. A space between the bulb 130 and a tube reflector 140 generally defines the germicidal chamber 136. The tube reflector 140 may suitably include, comprise, or be formed of a reflective surface (e.g., aluminum, titanium, and the like) for increasing the intensity and/or density of distribution of the radiation within the germicidal chamber 136. Furthermore, the germicidal chamber 136 or the tube reflector 140 may suitably include a layer 141 of titanium dioxide (TiO₂). TiO₂ is a potent photocatalyst capable of decomposing organic compounds when activated by UV radiation. Specifically, UV radiation causes the formation of hydroxyl (—OH) free radicals which are efficient oxidizers of organic matter, such as microorganisms targeted by the device 100 of the present invention.

The layer 141 of TiO₂ can be disposed within the germicidal chamber 136. Moreover, the layer 141 of TiO₂ can be disposed on the housing 102 inner wall surface of the tube reflector 142, thereby generally encircling the UV radiation source 104. As previously mentioned, the UV radiation source 104 includes a UV bulb 130 that omnidirectionally emits UV radiation. Accordingly, the disposition of the TiO₂ layer 141 about the bulb 130 generally maximizes UV radiation contact with the TiO₂ layer 141, and thereby maximizes photocatalysis of the TiO₂ layer 141. The maximization of photocatalysis suitably translates into a maximization of germicidal effectiveness. It is to be appreciated that TiO₂ layer 141 may suitably be applied to a replaceable filter pad or a screen or made integral with the tube reflector 140.

Returning to a discussion of the UV radiation source 104, the source 104, particularly the socket 132 and the bulb 130 thereof, can be communicably associated with the power supply 138 for delivering current thereto. The power supply 138 may be provided as a conventional AC or DC ballast or a high voltage high efficiency capacitor based power supply. The use of a DC ballast (coupled with a battery) can promote and enable the portability of the device 100. Such portability may enable use of the device 100 in any of a plurality of locations, such as on a boat, a car, a truck, a recreational vehicle, and the like.

With continued reference to FIGS. 4 and 5, the power supply 138 can be disposed at least partially within the housing 102, even more within the lower portion 114 and the projection 123 thereof. This position of the power supply 138 disposes it for ready communication with the powerplug assembly 122. Additionally, as previously mentioned, this area of the housing 102 includes the air inlet 128, which suitably contributes toward cooling of the power supply 138 by directing air thereover.

Returning now to a discussion of the UV radiation source 104 bulb 130, the bulb 130 can be removable for replacement. In one aspect, the bulb 130 threadably associates with the socket 132 for easy insertion and removal relative thereto. In another aspect, the bulb or UV light source is snappingly engaged with a socket or holder or any of various means that permit replaceability of the bulb. As best shown in FIG. 4, the top 117 of the upper portion 112 of the housing 102 may be suitably attached through a circumferential threaded portion 142. Removing the threaded portion 142 permits the top 117 to be removed, thereby enabling ready access to the bulb 130. In another embodiment, the entire upper portion 112 of the housing may be threadably attached to the lower portion 114. In this case, the upper portion 112 can suitably terminate at a position generally distal to the air outlet 116 in a circumferentially threaded face, and the lower portion 114 can suitably terminate at a position generally distal to the projection 123 in a circumferentially threaded face. The threaded faces of the upper and lower portions 112, 114 suitably interface in a conventional threaded manner and enable the upper and lower portions 112, 114 to be removably associated with each other through a screwing and/or unscrewing action.

The top 117 being threadably attached or secured suitably provides a safety feature. It is generally undesirable to permit direct exposure to active UV radiation, such as that encountered while current is being supplied to the bulb 130. In the configuration of the device 100 shown in FIGS. 1-5, the unscrewing of the threaded portion 142 generally requires that the device 100 be removed from a wall outlet. If the device 100 is so removed from the wall outlet, current cannot be supplied to the bulb 130, thereby eliminating a potential hazard of UV exposure. However, the device 100 may suitably also include various means (such as an appropriate interrupter) for preventing the flow of current to the bulb 130 if the upper portion 112, lower portion 114, and or top 117 become dissociated from the rest of the housing 102. This optionally provides a second safety feature.

In an alternate and not illustrated embodiment, the housing 102 may suitably include a removable panel that permits access to the bulb 130. The panel can be disposed along a vertical portion of the housing 102, even more along a midportion thereof that generally corresponds to the location of the germicidal chamber 136. In another embodiment, the panel can be suitably disposed along a semi-circumferential position that can be disposed generally above the powerplug assembly 122. In this embodiment, the panel can be maintained on the rear portion 108 of the device 100, and is accordingly maintained in proximity to the wall when the device 100 is plugged into a wall outlet. Such positioning of the removable bulb panel suitably requires that the device 100 be removed from the wall outlet prior to attempted bulb 130 removal, thereby preventing the flow of current to the bulb 130 and potential UV exposure.

The sterilization device 100 of the present invention may suitably also include various means for emitting illumination, thereby providing for a “night light” like effect, illumination of select portions of the environment in which the device 100 is positioned, and the like. Returning to FIGS. 1-5, the device 100 may suitably include an illumination ring 126 that is adapted to translate the UV radiation into visible and safe illumination. More specifically, the ring 126 is disposed at least partially circumferentially about the housing 102 and communicates between the housing 102 inner cavity 110 and the environment external to the device 100. Additionally, the ring 126 can be constructed of a generally transparent polymeric material. Accordingly, when the UV bulb 130 is active, the illumination ring 126 translates the UV bulb 130 illumination into a glowing effect that illuminates the ring 126.

The sterilization device 100 may suitably include various control features. In one aspect, the device 100 may suitably include a primary on/off switch 144 for selectably controlling actuation of the device 100. The primary switch 144 can be disposed on the housing 102 in a position that generally positions it for ready user access. The primary switch 144, if optionally present, can be provided as a conventional switch capable of selectably interrupting the flow of current to the UV radiation source 104. Alternatively, the device 100 may suitably not be provided with a switch, and thereby be active (i.e., deliver current to the UV radiation source 104) as long as the device 100 can be plugged into a wall outlet. In addition, an auxiliary switch 146 may be provided for activating or supplying power to the illumination ring 126. The auxiliary switch 146 may be depressed by pushing on a button or other control surface disposed generally along the top 117 or elsewhere on the housing 102. As seen in FIG. 5, a switch cover 145 can cover at least a portion of the primary switch 144.

A further objective of the present invention is to provide an air sterilization device capable of dispersing a pleasing fragrance or other desired scent. As shown in FIGS. 1-5, the device 100 further includes a scent receptacle 148 disposed in the top 117 for receiving a scented member 150. A plurality of apertures may be provided in the receptacle 148 to allow a portion of the airflow exiting the air outlet 116 to be diverted into and through the receptacle 148 containing the scented member 150. As the UV cleansed or decontaminated airflow enters the scent receptacle 148, air molecules in the airflow absorb or become laden with the scented chemicals that comprise the scented member 150. The scented member 150 may take the form of a liquid, gel, powder, solid or the like. In either case, the scented member 150 will slowly evaporate into the exiting air stream of the device 100 and be circulated throughout the environment in which the device 100 is operating. The receptacle 148 maybe be covered by a perforated or protective cover 152 to prevent tampering or spoilage of the scented member 150. It should be appreciated that the receptacle 150 may take on numerous configurations capable of retaining a scented medium. In addition, the receptacle 150 may be disposed anywhere along or in the housing 102 where the scented member 150 can be exposed to the airflow pathway of the device. Additionally, the scented member 150 can take the form of a plastic tablet having a shape that complements the shape of the receptacle 148 to allow for a resilient or snap fit of the scented member into the receptacle.

With reference to FIGS. 4 and 5A, the rotatable powerplug assembly 122 is shown in greater detail. Generally, the powerplug assembly 122 allows the device 100 to be used in a first use (e.g., a vertical orientation) or a second use position (e.g., a horizontal orientation) by rotating a portion of the assembly 122 in a clockwise or counterclockwise direction with respect to the housing 102. The powerplug assembly 122 includes a pair of prongs 160, a rotatable hub 162 and a retaining ring 164. As shown in FIG. 4, the rotatable hub 162 is confined between the rear portion 108 of the housing 102 and the retaining ring 164 which is rigidly attached to the rear portion 108. Naturally, an appropriate tolerance should exist between the rotatable hub 162 and the retaining ring 164 such that the hub 162 can rotate without binding against the retaining ring 164 or the rear portion 108.

With reference once again to FIG. 5A, the hub 162 includes a plurality of detents or nubs 166 disposed about an outer circumference of the hub 162. In the illustrated embodiment, two of a total of four (4) detents 166 are shown spaced approximately 90 degrees apart from one another. Of course, more or less detents could be used and their locations can be varied. The detents 166 are generally located along a weakened section 168 which serves as a biasing member for resisting a rearward movement of the detents 166. In the present embodiment, the weakened section or biasing member 168 is created by removing material along a rear portion of the hub directly behind the detent 166 and providing a slot 170 along a circumference of the hub 162 located radially inward from the detents 166. This permits the detent to elastically deflect in a forward to rearward direction (coincident with the axis of rotation of the hub 162). It should be appreciated that the weakened section or biasing member 168 could take on any suitable form such as a spring strip or coil.

As illustrated in FIG. 5A, the hub 162 is shown in the first use or vertical orientation such that the detents 166 align with a plurality of notches 172 formed along a thrust face 174 of the retaining ring 164. When the user of the device 100 desires to place the device 100 into the second use or horizontal orientation, the user simply grasps the prongs 160 (which are resiliently secured to the hub 162) and rotates the hub 162 with respect to the housing 102 in either the clockwise or counterclockwise direction. By way of example only, the hub 162 in FIG. 5A may be rotated towards the right in order to place the hub 162 in a second use position or horizontal orientation. As a torque is applied to the hub 162 to rotate it from the first or second use positions, the detents 166 will deflect in a rearward direction as the detents 166 ride out of the notches 172 and along the thrust face 174 of the retaining ring 164. When the respective detents 166 reach the adjacent respective notches 172, the detents 166 will be urged in a forward direction by the biasing members 168 and into the notches 172 to resiliently hold the device 100 in the desired orientation. To prevent the hub 162 from rotating greater than 90 degrees, a stop 176 can be provided with first and second stopping ends which correlate to the first and second use orientations. The stop 176 would interfere with a portion of the housing 102 once the first or second use position was reached. Thus excessive rotation of the hub and subsequent damage to an associated conductor used to establish an electrical connection between the prongs 160 and the power supply 138 would be prevented. In addition, a pair of mounting tabs 178 are provided for rigidly securing the retaining ring 164 to the rear portion 108 and an aperture 180 may be provided for one or more conductors to pass through and directly connect the prongs 160 to the power supply 138.

The sterilization device 100 may include various additional features that increase the air purification capabilities of the device 100. For example, the device 100 may include a HEPA filter (high efficiency particulate air filter), or other physical means (e.g., activated carbon, and the like) for removing particulates from an airflow.

It is to be appreciated that the sterilization device 100 of the present invention is not to be construed as limited to the foregoing discussion. It is to be appreciated that the present invention is generally directed to the concept of an air sterilizer, as substantially described hereinabove. Various housing and component orientation can be provided based on aesthetics without affecting the overall effectiveness of the device. Thus, the embodiments described herein are not limited to those shown in the Figures.

It is to be appreciated that additional modifications may be made to the invention without departing from the scope hereof. For example, the device may suitably be modified for relatively permanent association with a wall outlet. More specifically, in a hospital, hotel or similar environment, it may be desirable to secure the device to a wall outlet through a mechanical fastener (e.g., screw, etc.) or other means that significantly reduces the likelihood that the device will be inadvertently or otherwise removed therefrom.

Although the invention has been described with regard to certain preferred example embodiments, it is to be understood that the present disclosure has been made by way of example only, and that improvements, changes and modifications in the details of construction and the combination and arrangement of parts may be resorted to without departing form the spirit and scope of the invention. Such improvements, changes and modifications within the skill of the art are intended to be covered by the scope of the present disclosure.

1. An air sterilizer for eliminating an associated airborne contaminant from an associated volume of air, the air sterilizer comprising:
a housing including an air inlet and an air outlet;
an airflow pathway initiating approximately at the air inlet of the housing and terminating approximately at the air outlet of the housing;
an ultra-violet (UV) radiation source for producing a germicidal UV light, the UV source disposed in the airflow pathway of the housing for exposing the associated airborne contaminant to the germicidal UV light;
a reflecting member for increasing the exposure of the germicidal UV light to the associated airborne contaminant in the airflow pathway, the reflecting member being disposed in the housing proximal to the UV source;
a power supply for powering the UV source, the power supply disposed within the housing; and
an electrical power plug assembly secured to the housing and being in electrical communication with the power supply, the power plug assembly being selectively engageable with an associated electrical power receptacle, the power plug assembly capable of supporting the air sterilizer when the power plug is selectively engaged with the associated electrical power receptacle.

2. The air sterilizer of claim 1, further including a scented member disposed within the airflow pathway for dispersing a fragrance to the associated volume of air.

3. The air sterilizer of claim 2, wherein the scented member is a fragrance tablet disposed adjacent the air outlet of the air sterilizer.

4. The air sterilizer of claim 2, wherein the scented member is a non-heat activated scented plastic.

5. The air sterilizer of claim 1, wherein the reflecting member is one of an aluminum tube and a titanium tube that forms a conduit for a portion of the airflow pathway.

6. The air sterilizer of claim 5, wherein the reflecting member is coated with titanium dioxide (TiO₂).

7. The air sterilizer of claim 1, wherein the UV source includes at least one of an incandescent UV lamp, a fluorescent UV lamp, and a UV light emitting diode (LED).

8. The air sterilizer of claim 1, wherein the power plug assembly includes an electrical prong set for being selectively engaged with the associated electrical power receptacle, the prong set being rotatable with respect to the housing.

9. The air sterilizer of claim 1, wherein the power plug assembly includes an electrical prong set, a rotatable hub, and a retaining ring, the prong set being secured to the hub, the hub being rotatably engaged with the retaining ring, and the retaining ring being attached to the housing.

10. The air sterilizer of claim 9, wherein at least one of the hub and the ring includes one or more detents for engaging one or more respective notches in the other of the hub and the retaining ring, the detents including a biasing member for urging the detents into the respective notches when the hub is rotated from a first use position to a second use position.

11. A compact wall mountable air sterilization device for eliminating an associated airborne contaminant from an associated volume of air, the air sterilization device capable of being supported by an associated household electrical outlet comprising:
a body including a front, a rear, a top, an air inlet and an air outlet;
an airflow pathway initiating approximately at the air inlet of the body and terminating approximately at the air outlet of the body;
an ultra-violet (UV) radiation source for producing a germicidal UV light, the UV source disposed in the airflow pathway for exposing the associated airborne contaminant to the germicidal UV light;
a reflector disposed adjacent the UV source for increasing the exposure of the germicidal UV light to the associated airborne contaminant;
a fan disposed within the airflow pathway for accelerating the flow of the associated volume of air through the body;
a power supply for powering the UV source and the fan, the power supply disposed at the bottom of the housing;
a rotatable electrical power plug secured to the body and being in electrical communication with the power supply, the power plug being selectively engageable with the associated household electrical outlet, wherein the power plug is capable of supporting the body when the power plug is selectively engaged with the associated household electrical outlet.

12. The air sterilization device of claim 11, further including a scented member disposed adjacent the airflow pathway for dispersing a fragrance to the associated volume of air.

13. The air sterilization device of claim 12, wherein the body includes a recess disposed adjacent the air outlet and the scented member is a fragrance tablet received in the housing.

14. The air sterilization device of claim 12, wherein the scented member is a non-heat activated scented plastic.

15. The air sterilization device of claim 11, wherein a layer of titanium dioxide (TiO₂) is provided on a replaceable filter pad or a replaceable screen disposed within the airflow pathway.

16. The air sterilization device of claim 11, wherein the reflector comprises a hollow cylindrical tube defining at least a portion of the airflow pathway

17. The air sterilization device of claim 11, wherein the UV source produces a UV-C radiation.

18. The air sterilization device of claim 11, wherein the UV source includes a lamp configured to produce ozone (O₃).

19. The air sterilization device of claim 11, further including a plurality of baffles disposed about the airflow pathway for restricting the flow and increasing a dwell time of the associated volume of air through the body.

20. A compact wall mountable ultra-violet (UV) sterilizer for eliminating an associated airborne microorganism from an associated volume of air, the UV sterilizer capable of being supported by an associated household electrical outlet, the UV sterilizer comprising:
a housing including an air inlet and an air outlet;
an airflow pathway initiating approximately at the air inlet of the housing and terminating approximately at the air outlet of the housing;
an ultra-violet (UV) radiation source for producing a germicidal UV light, the UV source disposed in the airflow pathway of the housing for exposing the associated airborne microorganism to the germicidal UV light;
a tube reflector for increasing the exposure of the germicidal UV light to the associated airborne microorganism in the airflow pathway, the tube reflector being disposed in the housing proximal to the UV source and including a surface for receiving a UV activated layer of titanium dioxide (TiO₂);
a fan disposed within the airflow pathway for accelerating the flow of the associated volume of air through the housing;
a power supply for powering the UV source and the fan, the power supply disposed within the housing;
a rotatable electrical power plug secured to the housing and being in electrical communication with the power supply, the power plug being selectively engageable with an associated household electrical outlet, the power plug capable of supporting the air sterilizer when the power plug is selectively engaged with the associated household electrical outlet; and
a scent receptacle defined in the housing adjacent the air outlet for receiving a scented member for dispersing a fragrance into the associated volume of air when the air sterilizer is in an on state.
